(19) United States
(12) Patent Application Publication
(10) Pub. No.: US 2021/0181072 A1
(43) Pub. Date: Jun. 17, 2021

(54) DOUBLE TRENCH WELL FOR ASSAY PROCEDURES

(71) Applicant: LUMINEX CORPORATION, Austin, TX (US)

(72) Inventors: Colin BOZARTH, Austin, TX (US); Ross JOHNSON, Austin, TX (US); Adam SCHILFFARTH, Cedar Park, TX (US)

(73) Assignee: LUMINEX CORPORATION, Austin, TX (US)

(21) Appl. No.: 17/153,103

(22) Filed: Jan. 20, 2021

Related U.S. Application Data
(63) Continuation of application No. 14/838,647, filed on Aug. 28, 2015, now abandoned.
Application No. 14/838,647 is a division of application No. 13/401,522, filed on Feb. 21, 2012, now abandoned.
(60) Provisional application No. 61/446,918, filed on Feb. 25, 2011.

(51) Int. Cl. G01N 1/28; B01L 3/00; G01N 33/543

(52) U.S. Cl. CPC G01N 1/28; B01L 3/5085; G01N 33/54326; G01N 2500/00; B01L 2300/0829; B01L 2200/0668; B01L 2300/0851; Y10T 436/25

(57) ABSTRACT

Apparatuses, systems and methods for using assay preparation plates comprising wells with two trenches are presented. More specifically, well plates are presented that comprising an array of wells configured to retain a plurality of beads suspended in a fluid during an assay procedure, each well in the array comprising a first trench and a second trench, wherein the working volume of each well is between about 25 uL and about 10 mL.

### CROSS-REFERENCE TO RELATED APPLICATION

This application is a continuation of U.S. patent application Ser. No. 14/838,647, filed Aug. 28, 2015, which is a divisional of U.S. patent application Ser. No. 13/401,522, filed Feb. 21, 2012, which claims priority to U.S. Provisional Patent Application Ser. No. 61/446,918 filed Feb. 25, 2011. This provisional application is expressly incorporated by reference.

### BACKGROUND OF THE INVENTION

### I. Field of the Invention

This invention relates to assay preparation plates for use in an assay procedure and more particularly relates to apparatuses, systems and methods for using assay preparation plates comprising wells with two trenches.

### II. Related Art

Related art includes U.S. Patent Application No. 2009/0191638, U.S. Patent Application No. 2008/0075636, U.S. Patent Application No. 2007/0184463 A1, U.S. Pat. Nos. 5,779,907, and 5,897,783.

### BACKGROUND

Assay procedures may be used for a variety of purposes, including but not limited to biological screenings and environmental assessments. In some cases, a fluid may be processed prior to being analyzed to remove matter which is not of interest or which may conflict with obtaining accurate analysis results. In addition or alternatively, a fluid may be processed prior to being analyzed to offer results of greater sensitivity and/or specificity. Moreover, a fluid may, in some embodiments, be processed prior to being analyzed to convert the fluid into a form that is compatible with a particular analysis method, such as into a particle-based assay. The processing of fluid samples may be conducted manually, by using an automated “lab-on-a-chip” device, by using assay preparation modules, by using automated liquid handlers, by using plate washing devices, by using bead washing devices, or by using other suitable techniques.

In the wash step of a typical assay procedure, a plurality of magnetic beads (i.e., “microparticles”) are placed into a plurality of wells disposed on a well plate. A fluid is introduced into the plurality of well, forming a suspension that comprises the fluid and the beads. The suspension comprising the magnetic beads is introduced into a plurality of wells disposed on a well plate. A magnetic field is then applied to each well on the plate. This causes the magnetic beads to precipitate out of the suspension and form a mass (i.e., a “pellet”) near the source of the magnetic field. A volume of the fluid is aspirated from each well, ideally leaving the bead pellet in the well.

Various instruments (e.g., probes or pipettes) may be used to aspirate the fluid from the well. In some instances, the fluid is aspirated manually by an operator with a pipette, while in other cases, the fluid is aspirated automatically using an actuated probe. In each instance, the local velocity near the tip of the instrument is high. The high local velocity can strip beads from the pellet, re-suspending them in the fluid and causing them to be aspirated out of the well.

### SUMMARY OF THE INVENTION

Wells comprising two trenches and assay preparation plates comprising such wells are presented. Systems and methods for using such assay preparation plates are also presented. In general, the invention relates to assay preparation plates comprising double trench wells, systems comprising assay preparation plates comprising double trench wells, and methods of using the same.

Certain embodiments comprise a well configured to retain a plurality of beads suspended in a fluid during an assay procedure, the well comprising a first trench and a second trench, wherein the working volume of the well is between about 25 uL and about 10 mL. in some embodiments, the well may be substantially cylindrical and the first trench and the second trench may be concentric. The well may further comprise a ridge, wherein the first trench and the second trench are separated by a ridge. In other embodiments, the well comprises a ridge between the first trench and the second trench, wherein the first trench is parallel to the second trench.

Other embodiments comprise a well plate comprising an array of wells configured to retain a plurality of beads suspended in a fluid during an assay procedure, each well in the array comprising a first trench and a second trench, wherein the working volume of each well is between about 25 uL and about 10 mL. In certain embodiments, each well in the array is substantially cylindrical and the first trench and the second trench are concentric. In still other embodiments, each well in the array further comprises a ridge, wherein the first trench and the second trench are separated by the ridge.

Certain embodiments comprise a well plate in which each well in the array further comprises a ridge between the first trench and the second trench, and wherein the first trench is parallel to the second trench. The well plate may further comprise columns of wells arranged such that the first trench of a well is aligned with the first trench of at least one adjacent well in the same column. The array may comprise 48, 96, or 384 wells.

Still other embodiments comprise a system comprising: a well comprising a first trench and a second trench, wherein the working volume of the well is between about 25 uL and about 10 mL and a magnet external to the well adjacent to the first trench. In certain embodiments, the magnet may be an electromagnet or a permanent magnet.

Certain embodiments comprise a system comprising a well plate comprising an array of wells configured to retain a plurality of magnetic beads suspended in a fluid during an assay procedure, each well in the array comprising a first trench and a second trench wherein the working volume of each well is between about 25 uL and about 10 mL and a plurality of magnets external to the array of wells, where each magnet is adjacent to the first trench of one or more wells. In such embodiments, the magnet may be an electromagnet or a permanent magnet.

In certain embodiments, the well plate further comprises columns of wells arranged such that the first trench of a well is aligned with the first trench of at least one adjacent well in the same column. In some embodiments, the number of columns equals the number of magnets, and each magnet may be arranged such that the magnet is adjacent to the first trench of all the wells in one column. In other embodiments, the number of columns equals twice the number of magnets, and each magnet may be arranged such that the magnet is adjacent to the first trench of all the wells in two adjacent columns.

Still other embodiments comprise a method for collecting a sample of magnetic beads from a liquid, comprising obtaining a system comprising a well comprising a first trench and a second trench, wherein the working volume of the well is between about 25 uL and about 10 mL, and a magnet external to the well adjacent to the first trench, where the magnet is configured to selectively exert a magnetic force on the first trench; obtaining a first suspension comprising a plurality of magnetic beads suspended in a first liquid; introducing a volume of the first suspension into the well; exerting a magnetic force on the first trench; precipitating magnetic beads from the first suspension; forming a pellet of magnetic beads in the first trench; and aspirating a portion of the first liquid from the second trench.

In some embodiments, the method may further comprise removing the magnetic field from the first trench; obtaining a second liquid and introducing the second liquid into the first trench; and agitating the magnetic beads in the first trench to form a second suspension comprising the magnetic beads suspended in the second liquid.

The term “coupled” is defined as connected, although not necessarily directly, and not necessarily mechanically.

The terms “a” and “an” are defined as one or more unless this disclosure explicitly requires otherwise.

The term “substantially” and its variations are defined as being largely but not necessarily wholly what is specified as understood by one of ordinary skill in the art, and in one non-limiting embodiment “substantially” refers to ranges within 10%, preferably within 5%, more preferably within 1%, and most preferably within 0.5% of what is specified.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements possesses those one or more steps or elements, but is not limited to possessing only those one or more elements. Likewise, a step of a method or an element of a device that “comprises,” “has,” “includes” or “contains” one or more features possesses those one or more features, but is not limited to possessing only those one or more features. Furthermore, a device or structure that is configured in a certain way is configured in at least that way, but may also be configured in ways that are not listed.

Other features and associated advantages will become apparent with reference to the following detailed description of specific embodiments in connection with the accompanying drawings.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 illustrates one embodiment of a well plate.

FIGS. 2A-2C illustrate perspective, top, and side views of one embodiment of a well.

FIG. 3 illustrates one embodiment of a well plate.

FIGS. 4A-4E illustrate one embodiment of a method of bead washing.

FIGS. 5A-5B illustrate embodiments of system comprising a well plate and magnets.

FIGS. 6A-6B illustrate perspective and top views of one embodiment of a well.

FIGS. 7A-7B illustrate perspective and top views of one embodiment of a well.

FIG. 8 illustrates one embodiment of a method of bead washing.

FIG. 9 illustrates a schematic diagram of a method of bead washing using a conical well that was used in the investigative example.

FIG. 10 illustrates a schematic diagram of a method of bead washing using a double trench well that was used in the investigative example.

FIG. 11 is a chart illustrating bead retention performance of a conical well and a double trench well.

### DETAILED DESCRIPTION

Various features and advantageous details are explained more fully with reference to the nonlimiting embodiments that are illustrated in the accompanying drawings and detailed in the following description. Descriptions of well known starting materials, processing techniques, components, and equipment are omitted so as not to unnecessarily obscure the invention in detail. It should be understood, however, that the detailed description and the specific examples, while indicating embodiments of the invention, are given by way of illustration only, and not by way of limitation. Various substitutions, modifications, additions, and/or rearrangements within the spirit and/or scope of the underlying inventive concept will become apparent to those skilled in the art from this disclosure.

Washing is one step in an assay procedure. Examples of assay procedures may include a single-plex assay procedure, a multiplex assay procedure, and a bead coupling procedure in which an analyte is coupled to a bead, as well as other assay procedures. During the washing step, a suspension comprising a plurality of magnetic beads (i.e., “microparticles”) suspended in a fluid is introduced into a plurality of wells disposed on a well plate. As part of the washing step of the procedure, a magnetic field is applied to the suspension. This causes the magnetic beads to precipitate out of the suspension and form a pellet (i.e., a “mass”) near the source of the magnetic field. The fluid is aspirated from each well, leaving the beads in the well.

For example, the magnetic beads may comprise MagPlex™ Microspheres from Luminex Corp., 12212 Technology Blvd., Austin, Tex. 78727. MagPlex™ Microspheres are superparamagnetic carboxylated microspheres internally labeled with fluorescent dyes with magnetite encapsulated in a functional polymer outer coat containing surface carboxyl groups for covalent coupling of ligands. MagPlex™ Microspheres respond rapidly and efficiently to an applied magnetic field, but have negligible magnetic remanence, allowing rapid re-dispersion for further processing. The specifications for the MagPlex™ Microspheres are shown in Table 1 below:

TABLE 1
Specifications of MagPlex ™ Microspheres.

| | Specification |
|---|---|
| Package Configuration | |
| Microsphere Concentration (microspheres/mL) | 11.0 × 10⁶-14.5 × 10⁶ |
| Medium | <0.1% ProClin in Water |
| Microsphere Properties | |
| Median Microsphere Density (g/mL) | 1.10 ± 0.06 |
| Mode Microsphere Diameter (μm) | 6.5 ± 0.2 |
| RP1 background | ≤100 |
| Luminex100/200 Classification Efficiency | ≥80% |
| Luminex100/200 Misclassification | ≤2.0% |
| Luminex100/200 Doublet Discrimination Peak | 8000-15000 |
| Progenitor Microsphere Properties | |
| Median Microsphere Density (g/mL) | 1.10 ± 0.06 |
| Mode Microsphere Diameter (μm) | 6.5 ± 0.2 |
| Diameter Coefficient of Variation¹ | ≤5% |
| Functional groups | Carboxyl (COOH) |
| Iron Content | 2-4% |

¹Established from intermediate material, core particles

Various instruments (e.g., probes or pipettes) may be used to aspirate the fluid from the well. In some instances, the fluid is aspirated manually by an operator with a pipette, while in other cases, the fluid is aspirated automatically using an actuated probe. In each instance, the local velocity near the tip of the instrument is high.

In disclosed embodiments, the wells are configured to isolate or separate the location of the bead pellet from the point of aspiration. This lessens the effect of the high local velocity at the instrument tip on the bead pellet and decreases the number of beads that are stripped from the pellet and inadvertently aspirated from the well.

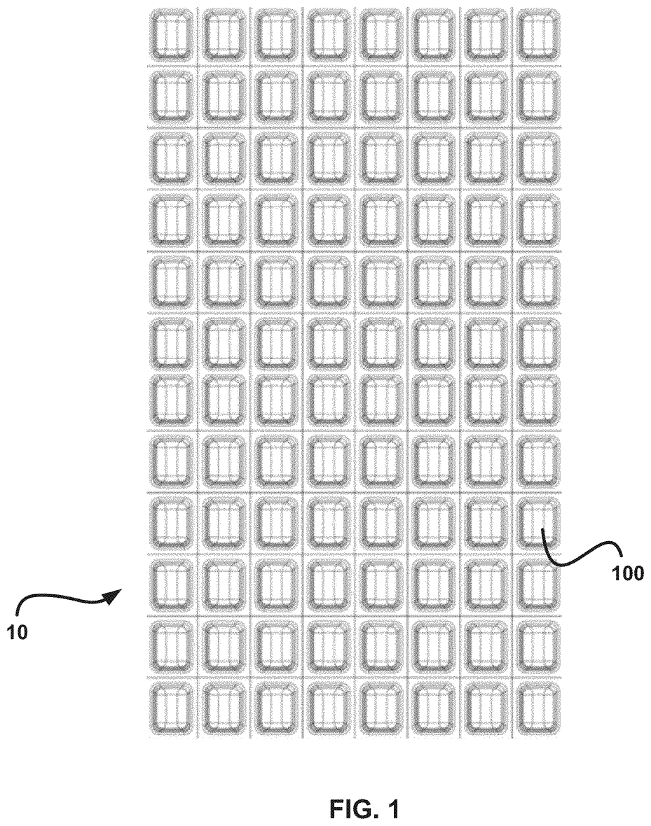

FIG. 1A illustrates one embodiment of a well plate 10 (i.e., “assay preparation plate”) comprising an array of ninety-six wells 100. The illustrated embodiment shows an array of eight columns and twelve rows of identical wells 100. In other embodiments, plates 10 may comprise an array of three hundred eighty-four wells. In still other embodiments, plates 10 may comprise arrays divisible by eight.

The working volume of each well 100 may range from about 25 uL to about 10 mL. In some embodiments, well plate 10 is configured to be used in an automated assay procedures; in such embodiments, the wells may be in the micro scale. In other embodiments, well plate 10 is configured to be used by a human user with a pipette, and in these instances, the wells may have a larger volume.

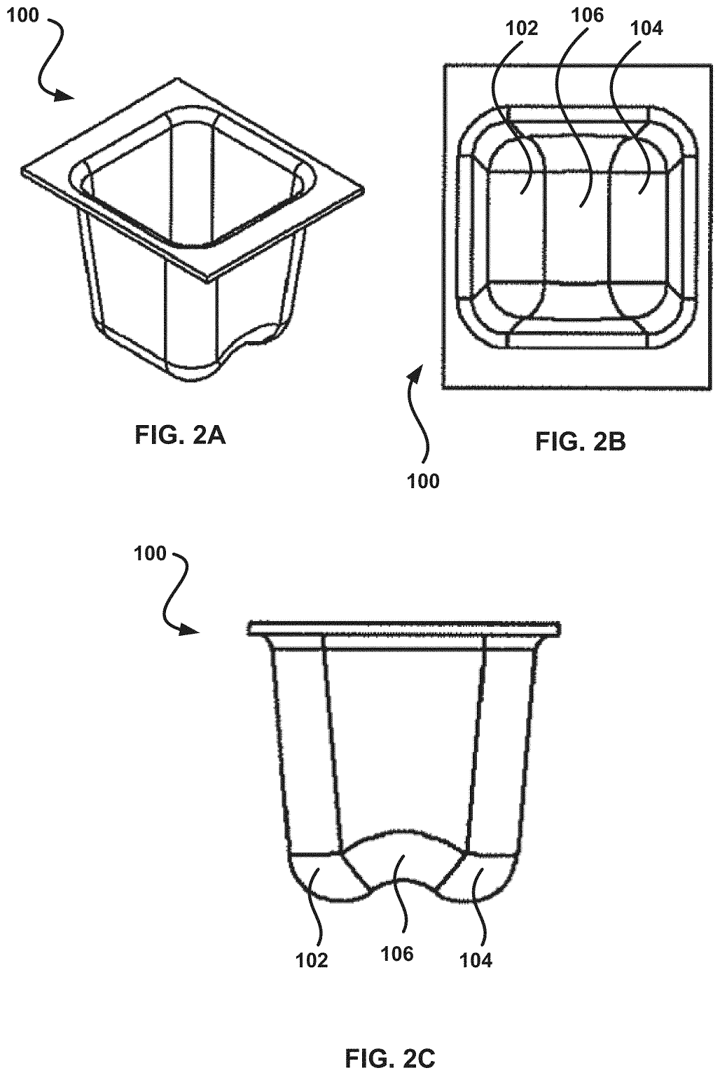

FIGS. 2A-2C illustrate perspective, top, and side views, respectively, of one embodiment of a well 100 (i.e., a “double trench well”). The illustrated embodiment of well 100 comprises a first trench 102 and a second trench 104. First trench 102 and second trench 104 are parallel to one another and are separated by a ridge 106.

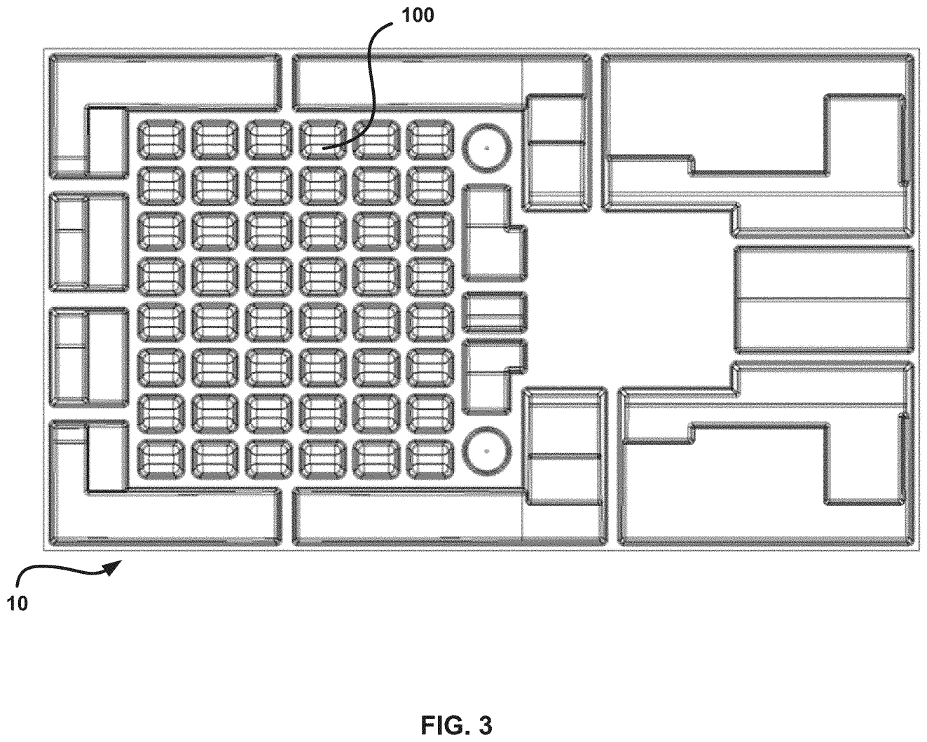

In some embodiments, such as those shown in FIG. 1, all the wells 100 on a well plate are substantially-identical double trench wells 100. In other embodiments, as shown in FIG. 3, well plate 10 may comprise double trench wells 100 and wells having other shapes. FIG. 3 illustrates an embodiment of a well plate 10 comprising an array of forty-eight wells 100, in addition to round wells and L-shaped wells.

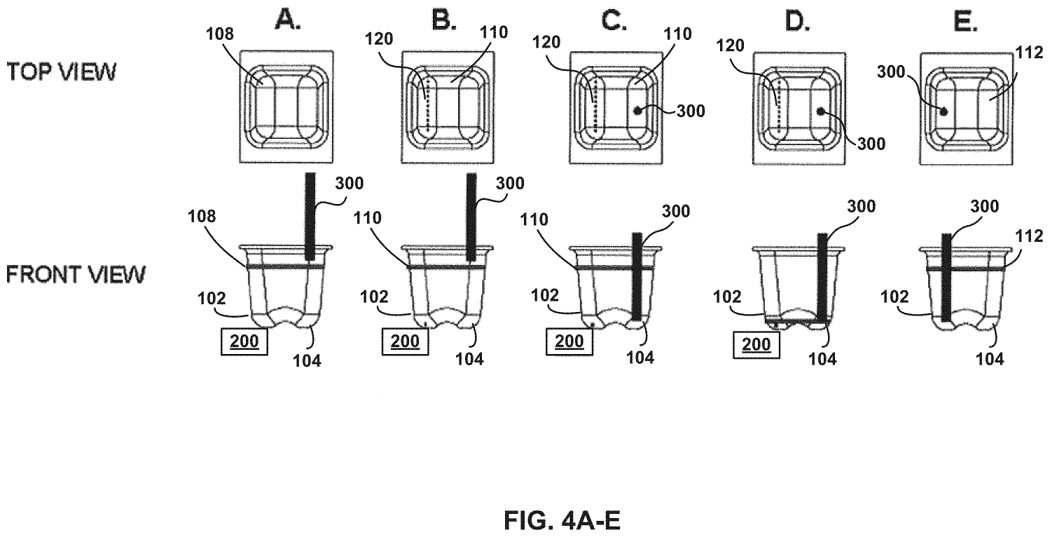

FIG. 4 illustrates a method for using and embodiment of well 100 to wash the plurality of magnetic beads. A plurality of magnetic beads are first placed in well 100. In step (A), a volume of first liquid 110 is introduced into well 100, forming a first suspension 108. In some embodiments, first liquid 110 is introduced to well 100 using an instrument 300. In various embodiments, instrument 300 may comprise a probe, a pipette, or another suitable conduit.

In some embodiments the plurality of magnetic beads may be initially introduced to the well suspended in first liquid 110 as a first suspension 108. In other words, first suspension 108 may be prepared outside wells 100.

A magnet 200 (i.e., a magnetic field source) is used to selectively apply a magnetic field to first trench 102. In some embodiments, magnet 200 is an electromagnet; in such embodiments, providing an electric current to magnet 200 produces an electric field, while removing the electric current removes the electric field. In other embodiments, magnet 200 is a permanent magnet that may be configured to be moved relative to first trench 102. As magnet 200 is moved closer to first trench 102, the strength of the magnetic field increases; as magnet 200 is moved further away from trench 102, the strength of the magnetic field decreases.

One skilled in the art will understand that “first trench” and “second trench” are terms used to describe the position of magnet 200 relative to each trench. First trench 102 is the trench adjacent to (i.e., closer to) magnet 200 in which pellet 120 is formed, while second trench 104 is the trench that is not adjacent to (i.e., farther from) magnet 200 and from which liquid 110 is aspirated.

As shown in step (B) of FIG. 4, magnet 200 applies a magnetic field to first trench 102. The magnetic field causes the magnetic beads suspended in first suspension 108 to precipitate out of first suspension 108 to form a pellet of beads 120 and a first liquid 110. Pellet 120 is formed in first trench 102 adjacent to magnet 200 and first liquid 110 fills the remainder of well 100.

Linear agitation or hydraulic agitation may be used in various embodiments. Instrument 300 may be moved within well 100 to agitate (i.e., stir) first suspension 108 in some embodiments. In other embodiments first suspension 108 may be agitated by vibrating or shaking well 100 or well plate 10 or by other known methods of agitation. In certain specific embodiments, the agitation frequency is 12 Hz for a duration of ten seconds. In other specific embodiments, the shaking amplitude may be between about 1 mm and about 4 mm.

In still other embodiments, hydraulic agitation may be used. Probe 300 may be used to perform a series of aspiration and dispense operations to create agitating fluid flow in well 100.

In some embodiments, first suspension 108 is agitated while the magnetic field is applied. While many beads form pellet 120 when the magnetic field is applied to well 100, some beads fail to migrate to the pellet formation site due interactions (such as friction forces or Van der Waals forces) with the well wall. So in some embodiments, suspension 108 may be agitated while magnetic field is applied to well 100 in order to dislodge beads from the wall of well 100 such that these beads migrate to the pellet formation site to form pellet 120.

In addition, one skilled in the art will understand that some number of magnetic beads may remain in suspension in first liquid 110. One skilled in the art will further understand that the ratio of beads that precipitates out of first suspension 108 may vary with the strength of the magnetic field and the duration that the magnetic field is applied. References to “a pellet” and “a supernatant” are not meant to limit the scope of the invention, but instead are adopted for the sake of clarity and ease of understanding.

As shown in step (C) of FIG. 4, instrument 300 is introduced into second trench 104, away from pellet 120 that has formed in first trench 102.

As shown in step (D) of FIG. 4, first liquid 110 is aspirated from second trench 104. By aspirating out of second trench 104, pellet 120 is undisturbed or minimally disturbed.

As shown in step (E) of FIG. 4, the magnetic field generated by magnet 200 is removed. In the illustrated embodiment, instrument 300 is placed into first trench 102. A second liquid is introduced into well 100 and agitated using instrument 300, which suspends the beads in the second liquid forming a second suspension 112. In other embodiments, the agitation may be by vibrating or shaking well 100 or well plate 10, or by other known methods of agitation.

The embodiment of well plate 10 depicted in FIG. 1 comprises eight columns and twelve rows of wells 100 arranged such that first trenches 102 of all wells 100 that are in the same column are aligned with one another. One magnet 200 may be configured to be adjacent to one, two, three, four, five, six, seven, eight, nine, ten, eleven, or twelve aligned first trenches 102.

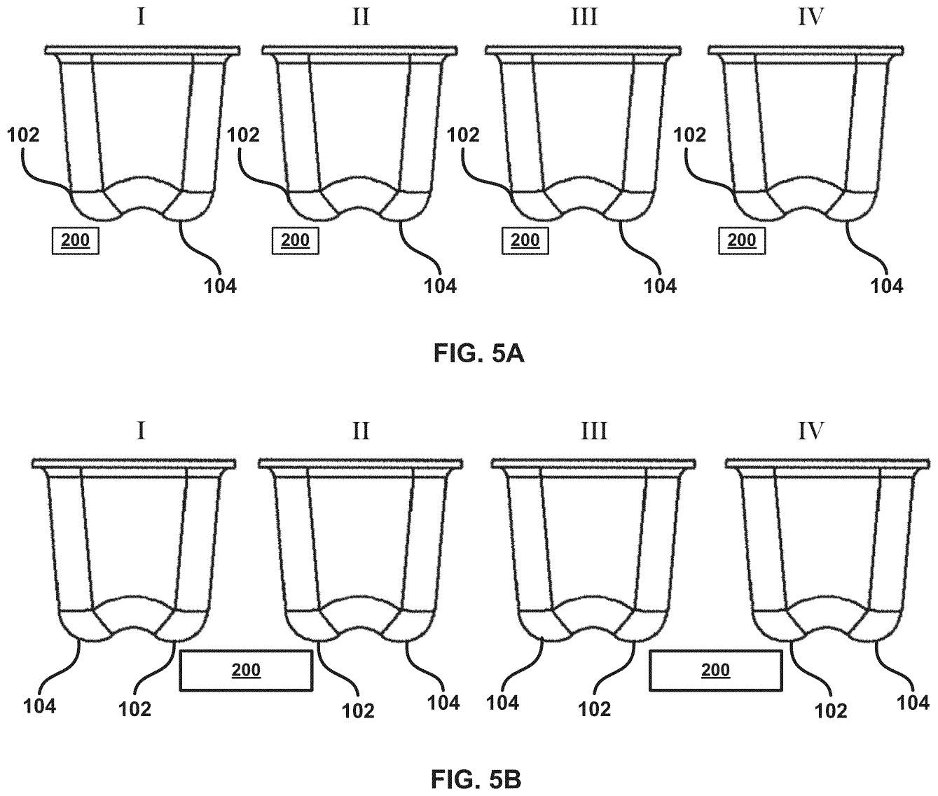

In certain embodiments of well plate 10, one magnet 200 may be configured to be adjacent to one or more first trenches 102 aligned in a column on a well plate 10. For example, FIG. 5A shows a side view of one row of four columns (I, II, III, IV) in an array of wells 100. The wells 100 are arranged such that each first trench 102 in a column is aligned with adjacent first trenches 102 in the same column. A magnet 200 extends the length of the columns such that it is adjacent to all the aligned first trenches 102 in a column. In the illustrated embodiment, the number of magnets 200 is equal to the number of columns—here, there are four columns and four magnets. Other embodiments may have a greater or fewer number of columns.

FIG. 5B shows another embodiment of a well plate 10. As in FIG. 5A, FIG. 5B illustrates a side view of one row of four columns (I, II, III, IV) in an array of wells 100. The wells 100 are arranged such that each first trench 102 in a column is aligned with adjacent first trenches 102 in the same column. A magnet 200 extends the length of the columns and is located between two columns, such that it is adjacent to all the aligned first trenches 102 in two adjacent columns. In the illustrated embodiment, one magnet 200 is adjacent to all first trenches 102 in column I and column II, and a second magnet 200 is adjacent to all first trenches in column III and column IV. In the illustrated embodiment, the number of magnets 200 is equal to half the number of columns—here, there are four columns and two magnets. Other embodiments may have a greater or fewer number of columns.

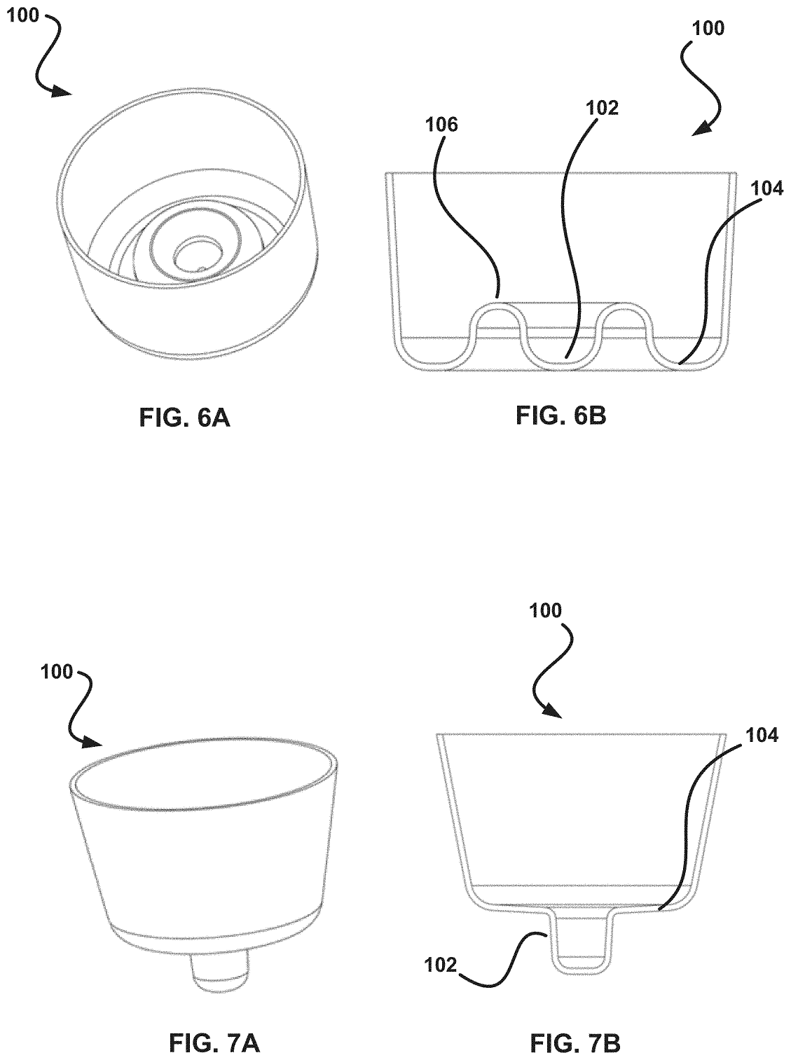

FIGS. 6A and 6B illustrate perspective and side views of another embodiment of well 100. In this embodiment, first trench 102 is circular and located in the center of well 100. Second trench 104 is concentric with first trench 102 and is separated from first trench 102 by ridge 106.

FIGS. 7A and 7B illustrate perspective and side views of still another embodiment of well 100. In this embodiment, first trench 102 is circular and is located in the center of well 100. Second trench 104 is concentric with first trench 102. First trench 102 extends to a greater depth than second trench 104.

The well embodiments shown in FIGS. 6A-7B may be configured and used substantially as discussed above in reference to FIGS. 1-5.

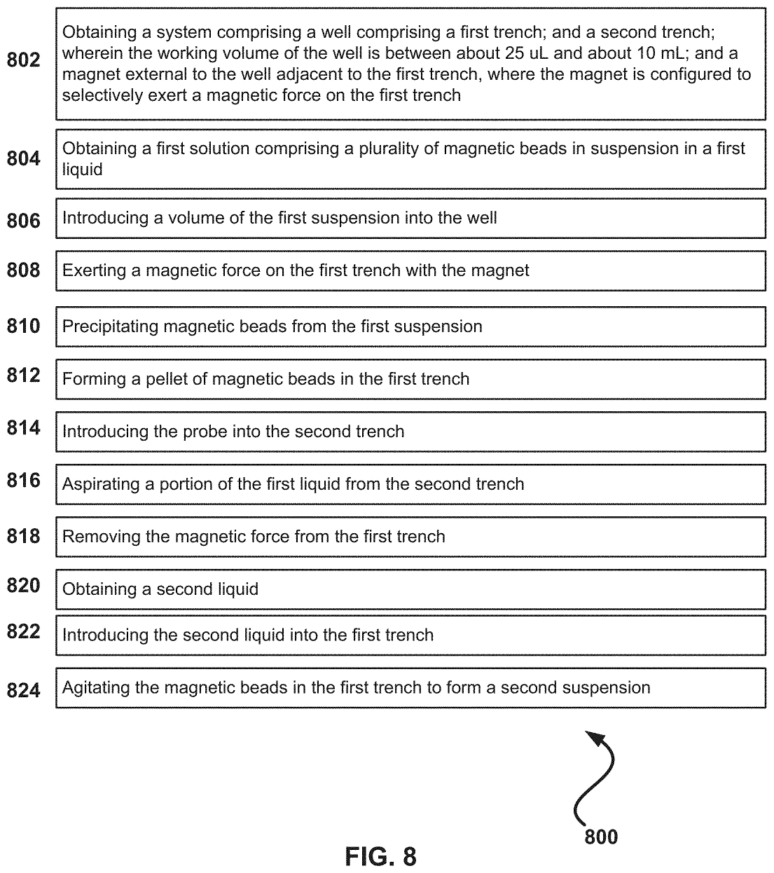

FIG. 8. illustrates one embodiment of a method of washing beads in a well.

### Investigative Example

Investigations were performed to examine the efficacy of the disclosed well; the results are discussed below.

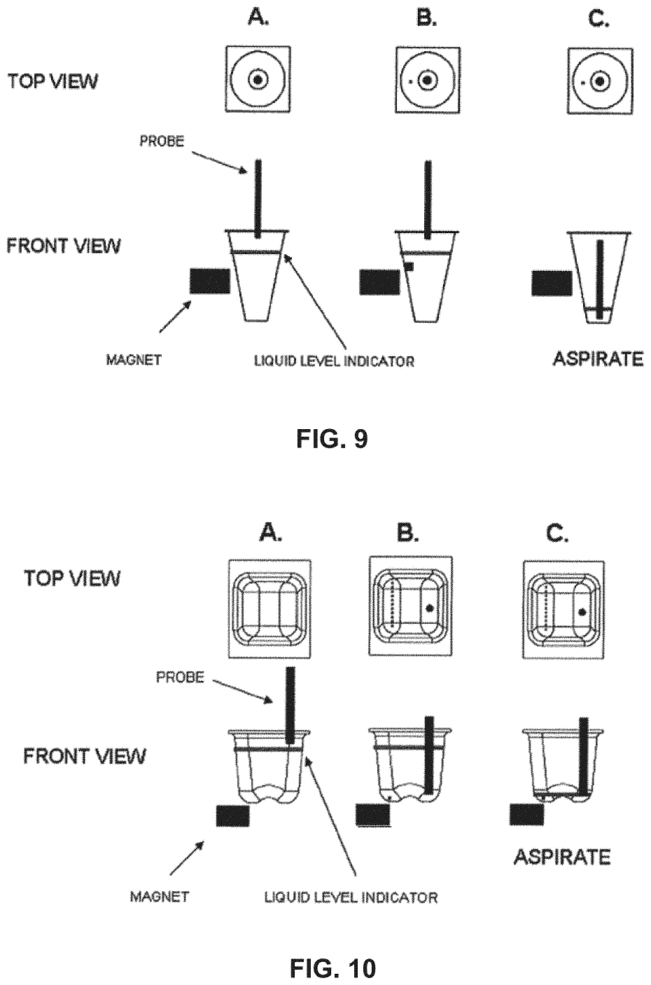

FIG. 9 illustrates three steps in a bead washing process performed with a conventional conical well. The process begins with beads being placed in a conical well. In step (A), a liquid is introduced in the conical well, suspending the beads in the liquid. A magnetic force is then applied adjacent to one side of the well. In step (B), the magnetic beads precipitate out of the suspension and form a pellet on the side of the wall nearest to the edge of the magnet. In step (C) the probe is introduced close to the bottom of the well and the supernatant is removed, leaving the beads in a pellet on the side wall. As the meniscus passes over the bead pellet the surface tension forces some of the beads to follow the meniscus moving the part of the pellet away from the magnet and down towards the probe. These stripped beads may then be lost during aspiration.

FIG. 10 illustrates the same three steps in a bead washing process, except this time, an embodiment of a double trench well is used. As in FIG. 9, the process begins with beads being placed in the first trench of the double trench well. In step (A), a liquid is introduced into the double trench well, suspending the beads in the liquid. A magnetic force is then applied adjacent to one side of the well. In step (B), the magnetic beads precipitate out of the suspension and form a pellet in the first trench nearest the magnet. In step (C) the probe is introduced into the second trench away from the magnet and the supernatant is removed, leaving the beads in a pellet in the first well.

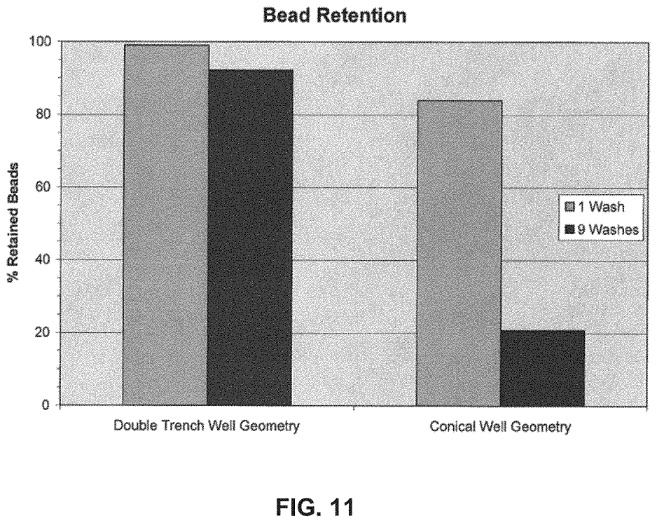

Steps (A) through (C) were repeated nine times. FIG. 11 is a chart comparing bead retention performance for the double trench well and the conical well after one wash and after nine washes. As can be seen, over 90% of the beads in the double trench well were retained after nine washes. In comparison, only about 21% of the beads in the conical well were retained after nine washes.

All of the methods disclosed and claimed herein can be made and executed without undue experimentation in light of the present disclosure. While the apparatus and methods of this invention have been described in terms of preferred embodiments, it will be apparent to those of skill in the art that variations may be applied to the methods and in the steps or in the sequence of steps of the method described herein without departing from the concept, spirit and scope of the invention. In addition, modifications may be made to the disclosed apparatus and components may be eliminated or substituted for the components described herein where the same or similar results would be achieved. All such similar substitutes and modifications apparent to those skilled in the art are deemed to be within the spirit, scope, and concept of the invention as defined by the appended claims.

We claim:

1. A method for collecting a sample of magnetic beads from a liquid, comprising:
(a) introducing into a well a suspension comprising a plurality of magnetic beads suspended in a liquid, the well comprising:
(i) a first trench;
(ii) a second trench; and
(iii) a ridge between the first trench and the second trench, wherein the first trench is parallel to the second trench, wherein the working volume of the well is between about 25 uL and about 10 mL;
(b) forming a pellet of magnetic beads in the first trench by exerting a magnetic force on the first trench;
(c) introducing a probe or pipette into the second trench; and
(d) aspirating with the probe or the pipette the liquid in the second trench while the pellet of magnetic beads is in the first trench.

2. The method of claim 1, further comprising removing the magnetic force from the first trench after aspirating the liquid from the second trench.

3. The method of claim 2, wherein the liquid is a first liquid, and further comprising introducing a second liquid into the well.

4. The method of claim 3, further comprising agitating the magnetic beads in the well to form a second suspension comprising the magnetic beads suspended in the second liquid.

5. The method of claim 1, wherein the magnetic beads have a mode diameter of 6.5±0.2 μm.

6. The method of claim 1, wherein the magnetic beads comprise carboxyl groups.
